(19) United States
(12) Patent Application Publication
(10) Pub. No.: US 2007/0265559 A1
(43) Pub. Date: Nov. 15, 2007

(54) Transfusion Safety Device

(75) Inventors: Takahiko Kunishige, Hiroshima-shi (JP); Naoki Miyamoto, Hiroshima-shi (JP)

Correspondence Address: WENDEROTH, LIND & PONACK, L.L.P., 2033 K STREET N. W., SUITE 800, WASHINGTON, DC, 20006-1021, US

(73) Assignee: JMS CO., LTD., Hiroshima (JP)

(21) Appl. No.: 11/662,338

(22) PCT Filed: Dec. 19, 2005
(86) PCT No.: PCT/JP05/23230

§ 371 Date: Mar. 8, 2007

(30) Foreign Application Priority Data
Dec. 20, 2004 (JP) 2004-368305

(51) Int. Cl. A61M 5/00

(52) U.S. Cl. 604/7

(57) ABSTRACT

A first object is to offer a safety device that allows to effectively solve problems regarding transfusion including free-flow issues in the case where the transfusion tube comes off from the transfusion pump, by employing a comparatively simple structure constructed at a low cost. A second object is to offer a safety device that can be newly added to an existing transfusion pump having no safety device. Specifically speaking, the case 100 in a tubular shape having a base, the compression spring 130, and the clamp 10 having the movable body 120 are set in the socket 20 having a cross-sectional shape of a square bracket. When the clamp 10 is set in the socket 20, the through-holes 101 and 121 become open, which allows distribution of fluid in the transfusion tube. On the other hand, when the clamp 10 becomes detached from the socket 20, the tension of the compression spring 130 causes the movable body 120 to slide, and the through-holes 101 and 121 are closed. As a result, the transfusion tube is blocked off, preventing the occurrence of a free flow.

### TECHNICAL FIED

The present invention relates to a transfusion safety device attached to a transfusion pump, in particular to improved technology for a safety mechanism operating when the transfusion tube comes off from the transfusion pump.

### BACKGROUND ART

At hospitals and medical facilities, transfusion systems are used in which a transfusion tube is put on a transfusion pump in order to send liquid medication, nutritional supplement, blood or the like to a patient or a solution sending line at predetermined speed and timing.

As the transfusion pump, a peristaltic pump is in wide use, as disclosed in Patent References 1 and 2. The peristaltic pump comes in a finger-type having multiple fingers (projections) and a roller-type having multiple cylindrical rollers. When the pump is in use, the transfusion tube is set so as to abut on the fingers or the rollers. At the driving, the peristaltic motion of the fingers or the rotational motion of the rollers firmly applies pressure in a manner that strokes the side surface of the transfusion tube to thereby squash and deform the transfusion tube. This causes peristaltic motion, which distributes liquid medication in the tube in a certain direction. In medical practices, the flow rate of the liquid medication in the transfusion tube is adjusted by appropriately setting the peristaltic motion of the fingers or the rotational speed of the rollers of the transfusion pump, its driving time, a drip rate on the drip chamber, the diameter of the transfusion tube, and the like, whereby the liquid medication is adequately administered to the patient or solution sending line.

Now then the transfusion pump has possibilities of the transfusion tube accidentally coming off from the transfusion pump. In such case, if the drive power of the transfusion pump ceases to work on the transfusion tube, there is a danger of causing a so-called free flow, i.e. a freely falling flow of the infusion under the force of gravity (a gravity fall).

To avoid the risk, Patent References 1 and 2 disclose a safety mechanism for preventing the occurrence of a free flow as a mechanism of the transfusion pump. Specifically speaking, a safety clamp is provided near a position where the transfusion tube is set up and the setup position is then protected by the pump door. In the case where the door is opened due to an error in operation or the like when the transfusion pump is being driven, the safety clamp is automatically activated to block off the transfusion tube so that the infusion is stopped.

<Patent Reference 1> Japanese Laid-Open Patent Application 2004-57577

<Patent Reference 2> Japanese Laid-Open Patent Application 2000-300667

### DISCLOSURE OF THE INVENTION

[Problems that the Invention is to Solve]

However, the operation of the safety mechanism for the transfusion pump described in Patent References 1 and 2 is designed based on the assumption that the transfusion tube is set to the transfusion pump, and has not been built by taking into account safety measures for the event in which the transfusion tube is completely detached from the transfusion pump. Therefore, the above mechanism of the safety clamp and the like does not operate in the case, for example, when a considerable pull force has been applied to the transfusion tube due to an operation mistake by a nurse or an unexpected accident and the transfusion tube has completely come off from the transfusion pump.

There is another problem related to the conventional safety mechanism: the safety mechanism is almost integrally built with the transfusion pump for structural reasons. Accordingly, in order to achieve the effects, purchasing a transfusion pump having the mechanism is necessary, and thus the safety mechanism cannot be newly added to a transfusion pump already in use in medical practices or a transfusion pump with no safety mechanism.

Given these factors, there is thought to be still room for improvement in the safety mechanism for the transfusion system regarding free-flow protection and the like.

The present invention has been made in view of the above issues, and has a first object of offering a transfusion safety device that allows to effectively solve problems regarding transfusion including free-flow issues in the case where the transfusion tube comes off from the transfusion pump, by employing a comparatively simple structure constructed at a low cost.

A second object is to offer a safety device that can be newly added to an existing transfusion pump having no safety device.

[Means to Solve the Problem]

In order to solve the above issues, the present invention is a transfusion safety device comprising a clamp attached to a transfusion tube and a socket in which the clamp is stored in a freely detachable manner. Here, the clamp has a structure in which fluid in the transfusion tube is released when the clamp is stored in the socket. When external force is applied to the transfusion tube, the clamp becomes detached from the socket. In the case when being detached from the socket, the clamp blocks off the distribution of the fluid by pressing the transfusion tube.

Here, the clamp may include: an insertion path into which the transfusion tube is inserted; and a movable mechanism that blocks off the distribution of the fluid by changing diameter of the insertion path.

In addition, the clamp may include: a case having a 1st through-hole and a hollow part that communicates with the 1st through-hole; a movable body which is slidably stored in the hollow part and has a 2nd through-hole; and a bias unit that slidably biases the movable body in the hollow part. Here, the movable mechanism causes the movable body to slide in the hollow part using the bias unit so that the 1st and 2nd through-holes overlap each other in an aperture direction thereof to form the insertion path, and changes the diameter of the insertion path by adjusting the degree of overlap between the 1st and 2nd through-holes.

To be more specific, the clamp may have a structure in which: (a) the 1st through-hole and another 1st through-hole, which is paired with the 1st through-hole, are positioned on a lateral face of the case in a tubular shape having a base, (b) the movable body having the 2nd through-hole is stored in the hollow part of the case together with the bias unit, and (c) the movable body slides in a longitudinal direction of the case, which results in changing (i) length of the clamp in a longitudinal direction thereof and (ii) the diameter of the insertion path. Here, the socket includes paired locking walls whose distance therebetween is smaller than the length of the clamp detached from the socket. The clamp further has a structure in which the movable body is pushed toward the case against the bias unit, and stored between the paired locking walls of the socket with the 1st and 2nd through-holes being open.

Note that the bias unit can be a spring.

In addition, the clamp may have one or more slits to guide the transfusion tube into an insertion path from a lateral side of the case.

Furthermore, the clamp can be stored in the socket with the slits facing the socket.

Additionally, the clamp may become detached from the socket in the longitudinal direction of the transfusion tube.

The present invention is also a transfusion pump set, in which the transfusion safety device above is attached to a transfusion pump.

Here, the socket may be attached to the transfusion pump with a higher strength than a pull force required to detach the clamp.

[Advantageous Effects of the Invention]

The transfusion safety device of the present invention having the structure above is a system in which the clamp and the socket are detachable from each other. Here, the clamp has a structure that limits the infusion volume of the transfusion tube when it is detached from the socket.

According to the structure, while the socket is fixed onto the transfusion pump, the transfusion tube is attached to the clamp, which is then stored in the socket. In this state of things, the transfusion tube is attached to the pump, whereby normal driving is performed. Here, in the event that the transfusion tube comes off from the transfusion pump, an external force is applied to the clamp as well as the transfusion tube, and these two become detached from the socket. This causes the clamp to operate so as to press the transfusion tube, which effectively prevents a problem of a free flow or the like.

Conventional safety devices for preventing a free flow described Patent References 1 and 2 are integrally incorporated with a transfusion pump as a component. Therefore, in the case when the transfusion tube becomes completely detached from the pump, there is no way to operate the safety mechanism. However, this problem can be fundamentally solved by using the transfusion safety device of the present invention with the transfusion pump.

In addition, the transfusion safety device of the present invention can be retrofitted to an existing transfusion pump in an attached manner by fixing the socket at a desired location on the pump using various kinds of fixing members, such as an adhesive tape, adhesive agent, or a screw. The transfusion safety device, thus, has significantly expanded versatility. Accordingly, there is no need to buy a new transfusion pump for obtaining the safety device, which realizes excellent cost performance. Furthermore, the structure is comparatively simple and thus the practicability is high.

### BRIEF DESCRIPTION OF THE DRAWINGS

FIG. 1 is a perspective view showing a structure of a transfusion safety device (clamp set) of Embodiment 1;

FIG. 2 shows a structure and operations of a clamp;

FIG. 3 shows a transfusion pump set using the clamp set;

FIGS. 4A and 4B show states of the clamp being attached and detached, respectively;

FIG. 5 shows a state in which the transfusion tube is detached from the transfusion pump;

FIGS. 6A, 6B and 6C show variations of the clamp;

FIG. 7 shows a structure of a clamp set of Embodiment 2; and

FIG. 8 shows a structure of a clamp set of Embodiment 3.

### EXPLANATION OF REFERENCES

MG1, MG2 magnet

1 transfusion safety device (clamp set)

2 transfusion pump

10, 11, 12, 13, 14 clamp

20, 25, 26, 27 socket

100, 106, 107, 108 case

102, 152 case slit

104 hollow part

122 movable-body slit part

123 stopper

120, 140, 150 movable body

130 spring

170 engaging part

171a, 171b split pin

172a, 172b bevel part

180 scissor-type clamp

181a, 181b paired arm

201, 202 locking wall

205 fixing wall

210 cover

221 socket fitting position

221a stage

221b depression

1811a, 1811b operation part

### BEST MODE FOR CARRYING OUT THE INVENTION

1. Structure of Transfusion Safety Device

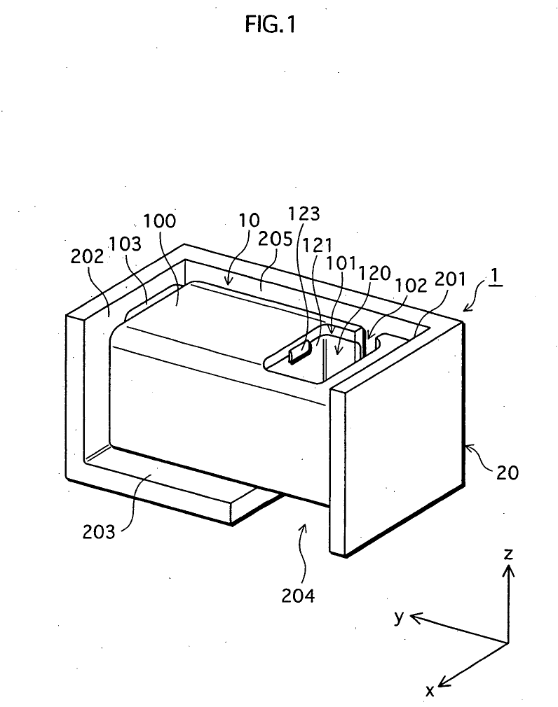

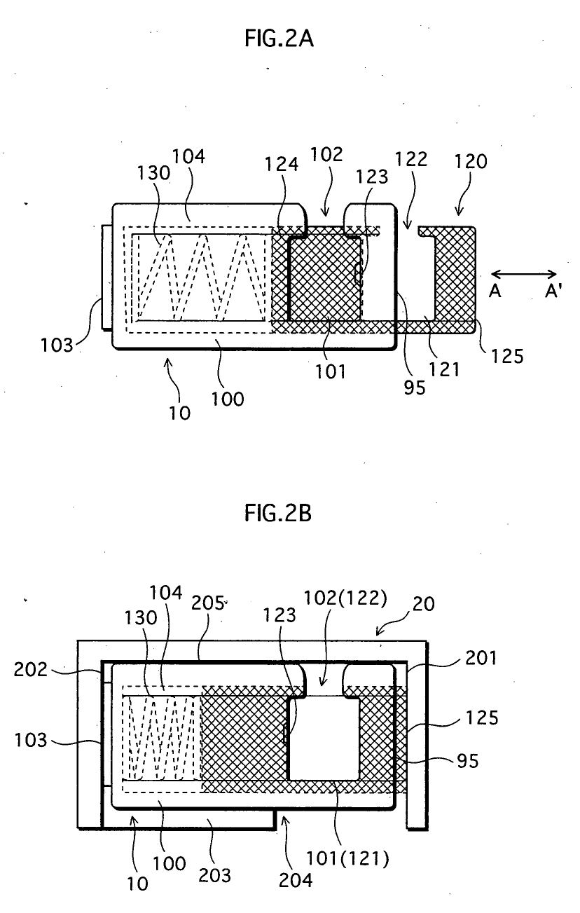

FIG. 1 shows a structure of a clamp set that is a transfusion safety device of Embodiment 1. FIG. 2 is given in order to explain the structure and operations of the clamp in detail.

Embodiment 1 is composed of a clamp 10 and a socket 20 housing therein the clamp 10, as shown in FIG. 1.

The socket 20 is formed by, for example, injection molding of a resin material. The socket 20 has a rectangle plate-like fixing wall 205 in the center, and a pair of locking walls 201 and 202 are located at both ends of the fixing wall 205 in the longitudinal direction (y direction) so as to form a square bracket shape with the fixing wall 205. Furthermore, a bottom wall 203 is located at the lower lateral side of the square-bracket body in a manner of extending from the locking wall 202 to the fixing wall 205. Herewith, within the socket 20, a rectangular-parallelepiped space is created by respective walls 201-203 and 205 surrounding thereof. A cutout 204 is provided between the bottom wall 203 and the locking wall 201 in accordance with through-holes 101 and 121 to be hereinafter described.

An example of the size of the socket 20 is 21 mm, 32 mm, and 16 mm in the x, y, and z directions, respectively, with a thickness of 5 mm.

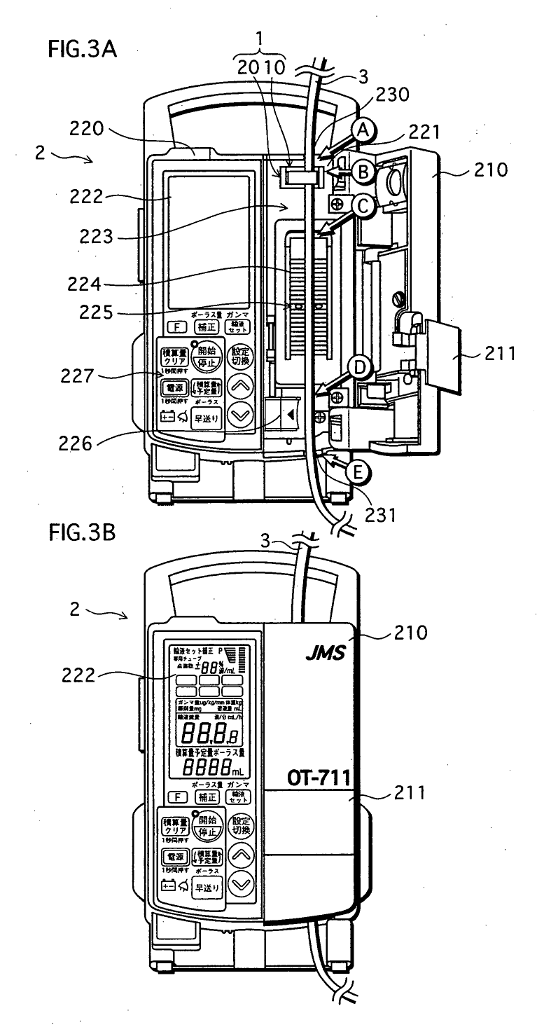

A two-sided tape or adhesive agent, which are examples of means to lock the socket 20 on the transfusion pump, is provided on the surface of the fixing wall 201 located at the back of the figure, and the socket 20 is retrofitted in a fitting position 221 of a transfusion pump 2 as shown in FIG. 3. The socket 20 should be fitted with strength enough to hold it in the case when the clamp 10 comes off therefrom (this case is to be hereinafter described). In other words, the socket 20 should be fitted with enough strength so as not to become disengaged from the transfusion pump together with the clamp 10. As the locking means, a hook, a hook and loop fastener, a screw can be also used. Furthermore, the socket 20 may be made from a material including a magnetic substance, and thereby can be locked using the magnetic force. As a matter of course, the fitting position in the transfusion pump has to be made of a metal or the like in the case when a magnetic substance is used.

FIGS. 2A and 2B are partially transparent views showing the structure and operations of the clam 10. According to the states of a compression spring 130, FIG. 2A shows the clamp 10 with the compression spring 130 being in a stretched state when the clamp 10 is disengaged from the socket; FIG. 2B shows the clamp 10 stored in the socket 20 (the compression spring 130 is being compressed). In FIGS. 2A and 2B, a movable body 120 is shown in hatching to facilitate visualization of its position within a case 100.

As shown in the figures, the clamp 10 includes the case 100, the movable body 120, the compression spring 130 and the like.

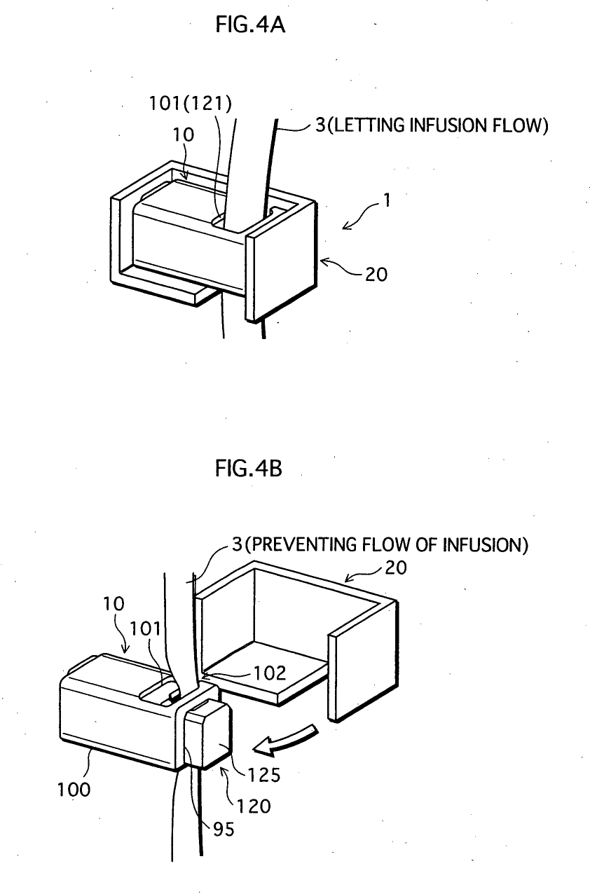

The case 100 is made of a resin material and has a hollow cylindrical body with a base. A through-hole 95 is provided at one end in the longitudinal direction, and has a case through-hole 101 at a location closer to the through-hole 95. This case through-hole 101 is used for inserting a transfusion tube 33 thereto (see FIG. 4). On the other end, a case end part 103 is formed and projects in a stepped manner so as to come into contact with the locking wall 202 of the socket 20. The cross section of the case 100 has a shape of a moderate square frame so as to facilitate the user to attach and detach the case 100 to and from the socket 20.

At the case through-hole 101, a slit 102 communicated with the outside is provided so that a transfusion tube can be attached from the lateral side of the case 100.

Additionally, in a hollow space 104 of the case 100, the compression spring 130 and the movable body 120 are sequentially stored, as shown in FIG. 2A.

The compression spring 130 is a coil spring made of a metallic material, such as stainless and nickel alloy. Within the hollow space 104 of the case 100, the compression spring 130 normally pushes up the movable body 120 to the side of the through-hole 95 with tension; when the clamp 10 is placed in the socket 20, however, the compression spring 130 is compressed at a predetermined pressure.

The movable body 120 is made of the same resin material used for the case 100, and has a rectangular parallelepiped shape with a contour set in the hollow space 104 of the case 100. On the lateral face, a movable-body through-hole 121, which has the same shape as the case through-hole 101, and a slit 122 are formed. According to the structure, the movable body 120 is provided so as to slidably move in the hollow part 104 of the case 100 in the longitudinal direction of the case 100 (A-A′ direction). The movable body 120 is normally biased by the compression spring 130 in a manner to move in the A′ direction; however, the movable body 120 moves in the A direction against the bias of the compression spring 130 when an external force is applied thereto-for example, when it is placed between the paired locking walls 201 and 202. When the movable body 120 has shifted in the A direction, the case through-hole 101 and the movable-body through-hole 121 coincide with each other, and thereby the insertion path of the transfusion tube 3 is obtained (see FIG. 2B. Contrarily, when the movable body 120 has shifted in the A′ direction, the diameter of the through-hole changes as a result that the case through-hole 101 and movable-body through-hole 121 become out of line, and the insertion path is closed (FIG. 2A)). At this point, the movable body 120 projects over the case 100, and the projected portion is a movable-body end part 125, which is symmetrical to the case end part 103.

In the socket 20, the distance between the locking walls 201 and 202 is arranged to be shorter than the length of the clamp 10 detached from the socket 20, and thereby a compression force is applied to the movable body 120 in the A direction when the clamp 10 is placed in the socket 20.

Note that a projected stopper (claw) 123 is formed at one end of the movable-body through-hole 121, and the stopper 123 locks with a peripheral part of the case through-hole 101 when the movable body 120 is shifted in the A′ direction, whereby preventing the movable body 120 from coming off from the case 100.

The clamp 10 and socket 20 may be made of a material other than a resin material, for example, a metallic material.

When being placed in the socket 20, the clamp 10 has the slit 102 face the socket 20 side. This is preferable because the transfusion tube 3 is effectively prevented from coming off from the clamp 10.

2. Fitting to Transfusion Pump and Effect of Clamp Set

FIG. 3 shows a structural example of a transfusion pump set comprising the transfusion pump 2, a clamp set 1, and the transfusion tube 3.

The transfusion pump 2 shown in FIG. 3 is a publicly known peristaltic pump, and a peristaltic roller is housed in a box-type case 220. On the front face of the case 220, a display unit 222, an operation unit 227, a pump interior, a transfusion set unit 223, a pump door 210, a door lever 211, and the like are provided. The case 220 also houses a control circuit (not shown) for receiving user information that is input from the operation unit 227 and driving the roller according to the setup.

On the display unit 222, the flow rate of infusion, infusion volume, drip rate, and various pump states (presence or absence of air bubbles, fluid dripping or not, door in an open or closed state, state of internal batteries, state of the block sensor, etc) are displayed, as shown in FIG. 3B, when the transfusion pump 2 is being driven.

In the operation unit 227, main buttons including a power button, a drive start/stop button, and a setting input button are provided along with buttons for adjustment, such as a memory clear button and a setting change button. Transfusion conditions input thereto is transmitted to a control circuit inside the case 220 and stored in a memory unit of a microcomputer in the circuit.

The pump door 210 is provided on the case 220 with a hinge, which allows the pump door 210 to be opened and closed freely. In addition, the pump door 210 covers the entire pump interior of the pump case 220, in which the transfusion tube 3 is set.

In the upper part of the pump interior, the back side of the locking wall 205 of the socket 20 is affixed using a two-sided tape or the like, and thereby the clamp set 1 of Embodiment 1 is set up in the transfusion pump 2.

On the downstream side from the clamp set 1, the transfusion set unit 223 housed in the case 220 is located. As to the transfusion set unit 223, a finger unit 224, which is composed of multiple disk-shaped projections and to which driving forces of the peristaltic roller is transmitted, is arranged so as to be decentered with respect to the driving axis and exposed to the outside, as shown in FIG. 3A. When the transfusion pump 2 is being driven, the pumping action is created by each of the decentered projections sequentially pressing the transfusion tube 3 and making peristaltic motion to firmly stroke the transfusion tube 3.

In the center of the finger unit 224, a clip-type block sensor 225 is provided to hold the transfusion tube 3.

On the downstream of the transfusion set unit 223, a door safety clamp 226 is provided. This is a structure for, when the pump door 210 opens for some reason during the transfusion pump 2 being driven, holding the transfusion tube 3 so as not to come off from the transfusion set unit 223.

Additionally, on the pump case 220, grooves 230 and 231 are provided for guiding the transfusion tube 3.

According to the clamp set with the above structure, the user (e.g. a nurse) first attaches the transfusion tube 3 to the transfusion pump 2 before use. An example of the attachment order is shown in FIG. 3A from A to E: the groove 230, the clamp 10, the transfusion set unit 223, the door safety clamp 226, and then the groove 231.

Here, the attachment of the transfusion tube 3 to the clamp 10 is made by inserting the transfusion tube 3 into the through-holes 101 and 121 of the clamp 10. As shown in FIG. 1, providing the slits 102 and 122 on the clamp 10 allows the clamp 10 to be set in place without need of hauling in the end of the transfusion tube 3 and inserting it into the through-holes 101 and 121, which improves the operationality. After the transfusion tube 3 is attached to the clamp 10, the clamp 10 is placed in the socket 20.

After the completion of the attachment, the user closes the pump door 210 and locks the pump door 210 being in a closed state using the door lever 211. While maintaining the state, the user drives the transfusion pump 2 (the state shown in FIG. 3B). Here, FIG. 4A shows the condition of the clamp set 1 corresponding to the state of FIG. 3A. The clamp 10 is placed in the socket 20, and this state allows the liquid medication in the transfusion tube 3 to flow therethrough.

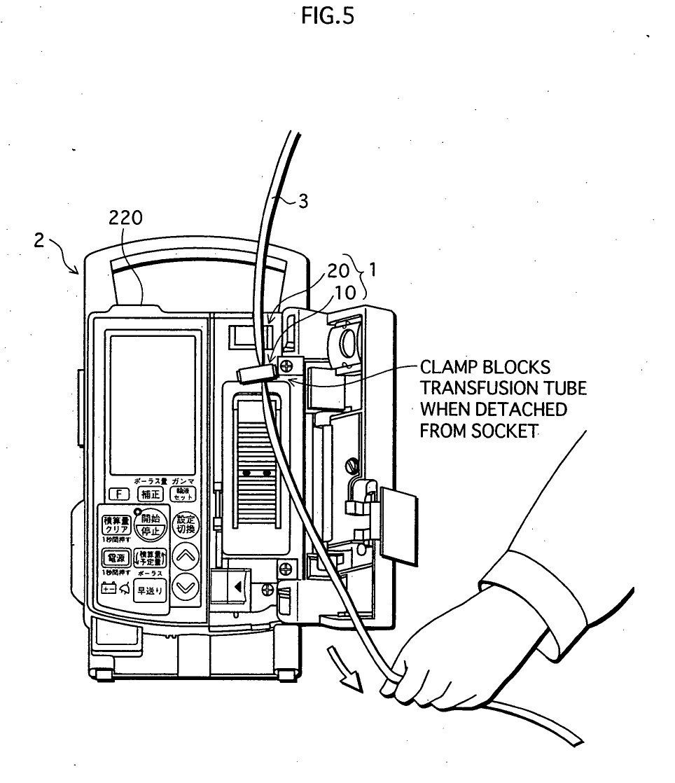

In the case when the transfusion tube 3 is pulled as a result of some strong force being applied due to the nurse's erroneous operation (specifically speaking, forgetting to close a clench attached to the transfusion tube) or from any cause such as application of unexpected stress, the pump door 210 opens as shown in FIG. 5, and the clamp 10 comes off from the socket 20. Here, if the pull force is relatively low and the transfusion tube 3 remains fixed to the safety clamp 226, the clamp 226 operates as the pump door 210 opens, which leads to actuation of the safety mechanism that blocks the transfusion tube 3, as in the conventional mechanism. However, according to the conventional mechanism, if the transfusion tube 3 is detached also from the safety clamp 226, a safety device provided on the transfusion pump 2 does not operate, causing a problem of a free flow or the like.

According to Embodiment 1, therefore, when the clamp 10 is detached from the socket 20, as shown in FIG. 4B, due to relatively large pull force, the movable body 120 sticks out from the through-hole 95 of the case 100 due to the bias of the compression spring 130, and the movable-body through-hole 121 moves relatively to the case through-hole 101. Then, the transfusion tube 3 is pinched at the sides and deformed under pressure. With this operation, the inside of the transfusion tube 3 is blocked off, and thus a (stopper) mechanism for preventing the flow of infusion is realized. Namely, Embodiment 1 is capable of offering high performance for effectively preventing a problem of a free flow and the like even after the transfusion tube 3 has been completely detached from the transfusion pump 2.

The clamp set 1 of Embodiment 1 has expanded versatility because of not depending on the structure of the transfusion pump, and has an advantage of being able to be attached to an existing transfusion pump in an attached manner at a desired location by a two-sided tape, adhesive agent, or screw. Therefore, the clamp set of the present invention can be provided to almost all types of existing transfusion pumps in an attached manner, and therefore eliminates the necessity of newly buying a transfusion pump with a safety device, as in conventional cases. Thus, the clamp set of the present invention utilizes an existing equipment effectively, enabling excellent cost performance.

Application of the clamp set 1 of the present invention is not limited to the transfusion pump 2; the clamp set 1 can be applied also to a liquid medication administration system and a transfusion set which is used, for example, for intravenous drip and includes a transfusion bag, a clench, a transfusion tube, a drip chamber and the like.

3. Variations in Transfusion Device

The clamp set of the present invention, which serves as a transfusion safety device, is not limited to the structure of Embodiment 1, and can take another structure as long as the clamp is detachable from the socket, and blocks the transfusion tube when detached from the socket.

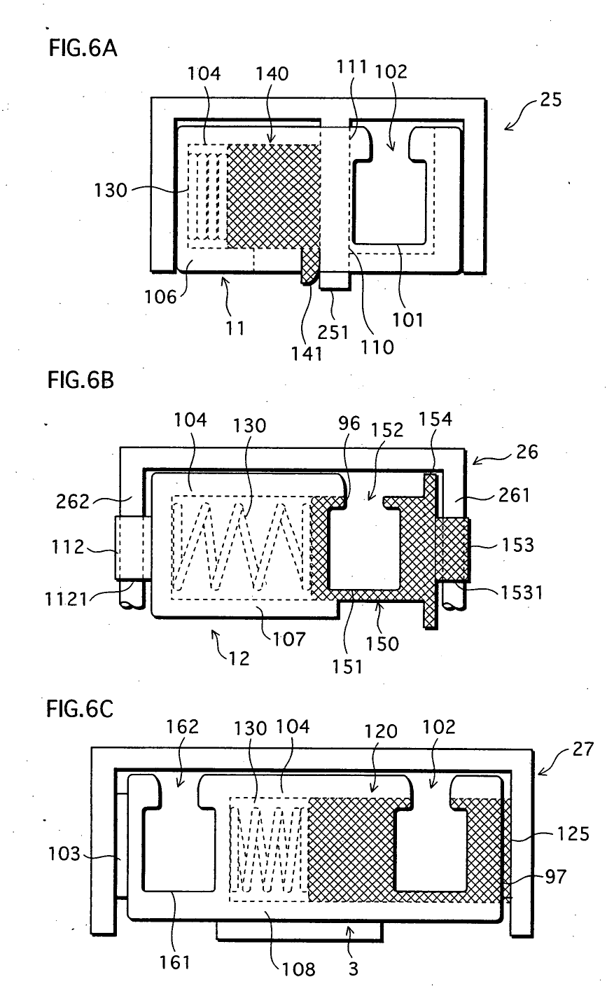

FIG. 6 shows variations of the clamp set of Embodiment 1.

FIG. 6A shows a structural example (Variation 1) that locks the clamp by a part of the socket penetrating into the clamp.

Although having basically the same structure as the clamp 10, a clamp 11 of Variation 1 is characterized by being provided on the middle of the lateral face of the case 106 along the longitudinal direction and communicates with the hollow part 104, and a rectangular-parallelepiped movable-body 140 is placed in the case with a tab 141 exposed.

In order to place the clamp 11 in a socket 25, the user pushes down the tab 141 along the slit 111 and thus compresses the compression spring 130, and inserting a rod-like projection 251 of the socket 25 into a hole 110 and the slit 111. Thereby, the clamp 11 is attached to the socket 25. With this type of clamp 11, similar effects to that of Embodiment 1 can be realized by the movable-body 140 sliding inside the clamp 11 with the use of the guide mechanism of the slit 111 and tab 141.

The spring housed in the clamp case is not limited to the structure that pushes out the movable-body to the outside when the spring has the rest length. FIG. 6B shows a structural example (Variation 2) in which a spring is biased in the compression direction and becomes stretched when the clamp is placed in the socket.

In Variation 2 shown in the figure, a case 107 of a clamp 12 and a movable-body 150 have through-holes 1121 and 1531, respectively, and a socket 26 has a pair of shaft locking units 261 and 262, which have been made to fit to the diameters of the trough-holes 1121 and 1531.

The movable-body 150 is connected to the compression spring 130 in the hollow part 104 inside the case 107, and is normally biased in the compression direction inside the case 107 by the contraction force of the compression spring 130. On the movable-body 150, a brim 154 is formed at the end, and the movable-body 150 is normally hidden in the case completely due to the contraction of the compression spring 130 and the through-hole 151 is closed. On the other hand, when placed in the socket 26, the pair of shaft locking units 261 and 262 are inserted into the through-holes 1121 and 1531, and the through-hole 151 is open as shown in FIG. 6B. According to the structure, similar effects to that of Embodiment 1 can be realized. In addition, Variation 2 also has a merit of contributing to downsizing since the clamp being detached from the socket 20 has a shorter length than when it is placed in the socket 20.

Furthermore, the number of through-holes provided on the clamp is not limited to one and can be more than one. Variation 3 shown in FIG. 6C is characterized in that another through-hole 161 and slit 162 are provided on a case 108 of a clamp 13, which is placed in a socket 27. Other than this point, the clamp 13 has the same structure as the clamp 10 of Embodiment 1.

According to Variation 3, besides the effects of Embodiment 1, the attachment method of the transfusion tube 3 can be changed because of the through-holes 161 and slit 162 being provided. Namely, when attached to the transfusion pump 2 shown in FIG. 3, the transfusion tube 3 can be attached using the slit 102. On the other hand, when the transfusion tube 3 cannot be extended in the up and down directions of the pump due to, for example, constraints on the fitting position to the transfusion pump and the shape of the transfusion pump, the transfusion tube 3 can be folded in a U-shape using the through-hole 161 and slit 162. With such an idea, Variation 3 excels in handling the transfusion tube 3.

Note that, when Variation 3 is applied, attention must be paid so as not to break the tube and block off the inside of the tube due to folding the transfusion tube 3.

4. Other Particulars

In Embodiment 1, the clamp set is located on the upstream side with respect to the fitting position of the transfusion pump; however, it can be set on the downstream side of the transfusion pump. In addition, more than one clamp may be attached to one transfusion tube.

In Embodiment 1, the transfusion tube has a structure that allows blood or liquid medication to flow therethrough. It is a matter of course, however, that the infusion is not limited to these, and the transfusion tube may have a structure that allows other solutions, such as water and physiological saline water.

### EMBODIMENT 2

The following describes Embodiment 2 of the present invention, with a focus on differences from Embodiment 1.

The structure of the clamp set of Embodiment 1 above is formed by taking into account mainly the case that the transfusion tube 3 accidentally comes off from the pump when the transfusion tube 3 is unexpectedly pulled during the pump being driven. The present invention is however not limited to such a case, and the clamp may be set in operation when the cover 210 is unnecessarily opened.

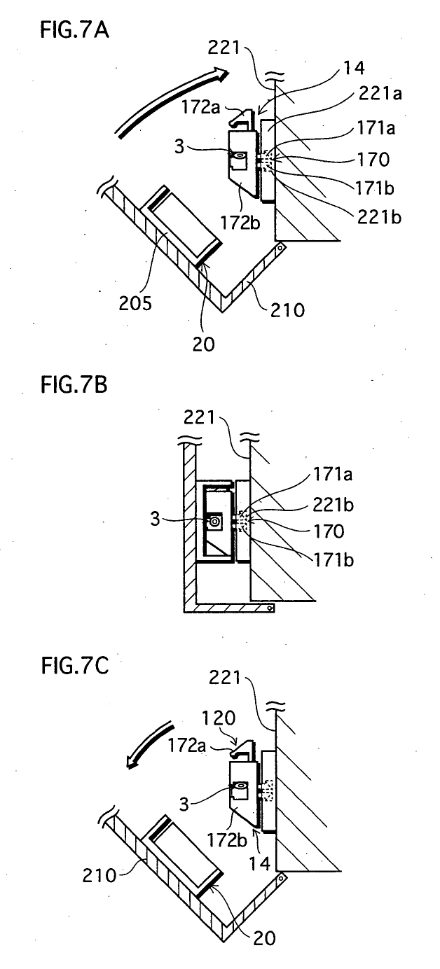

FIG. 7 is a partially enlarged figure showing a structure around a socket fitting position 221 of Embodiment 2. The figure shows a pump looked down from the top. Embodiment 2 has a structure in which the clamp set is fitted to the back side of the cover 210.

The clamp 14 is largely the same as the clamp 10; however, the clamp 14 has, as a first engaging part, an engaging part 170 comprising split-pin type projections 171a and 171b on the lateral side at a location facing the socket fitting position 221. On the other hand, at the socket fitting position 221 facing the engaging part 170, a stage 221a is provided as a second engaging part having a depression 221b which reversibly engages with the engaging part 170. The strength of the engagement between the engaging part 170 and the depression 221b is set larger than the strength required disengaging the clamp 14 from the socket 20.

In addition, an oblique angle is partially formed, on both end parts of the clamp 14 in the longitudinal direction, at locations adjacent to the socket 20, and bevel parts 172a and 172b are provided. These allow the clamp 14 to fit quite smoothly into the socket 20.

According to the structure above, the user first attaches the transfusion tube 3 to the clamp 14, and sets these on the stage 221a. At this point, the engaging part 170 of the clamp 10 engages with the depression 221b of the stage 221a, and a stable setup is obtained (FIG. 7A). In this state, the cover 210 is closed, and then the clamp 14 is automatically attached to the socket 20 in a quite smooth manner due to the action of the bevel parts 172a and 172b (FIG. 7B). Herewith, the transfusion condition of the transfusion tube 3 becomes free.

In this state, if the cover 210 becomes unexpectedly opened, the socket 20 moves with the cover 210. On the other hand, the clamp 14 becomes detached from the socket 20 due to the engagement effect of the engaging part 170 and the depression 221b and remains on the pump side. Then, due to the release of the clamp 14 from the socket 20, the movable body 120 projects from the clamp 14 and the transfusion tube 3 is blocked off (FIG. 7C).

According to Embodiment 2 performing the above operations, effects almost similar to those of Embodiment 1 can be realized. Moreover, the occurrence of a free flow can be prevented by blocking off the tube 3 in conjunction with the opening of the cover 210 even if a direct pull force on the transfusion tube 3 is not exerted. As a result, Embodiment 2 offers higher safety level as compared to Embodiment 1.

In addition, Embodiment 2 can be realized by attaching the stage 221a and clamp set 1 to a common transfusion pump at predetermined positions thereon, and thus has significantly expanded versatility. Note that the depression 221b may be provided directly on the body of the pump without the stage 221a.

After the engagement, the clamp 14 can be easily detached by pushing down the split pins 171a and 171b from the sides or tilting the clamp 14 with respect to the stage 221a.

It is a matter of course the structure of the engaging part 170 using the split pins 171a and 171b is one example, and any structure can be used as long as the engagement effect is realized so that the clamp is fixed onto the stage side when the cover is opened. For example, the above effect can be achieved by providing magnets, a hook and loop fastener, two-sided tapes, or the like to the clamp and the stage.

Additionally, FIG. 7 shows the structure in which the socket 20 is provided on the internal face of the cover 210; however, the position of the socket 20 is not limited to this case. For example, the same effects above can be obtained by fitting the socket on the upper lateral side of the cover 210. Here, the structure is designed so that the clamp 14 becomes engaged, in accordance with the position of the socket, by the engaging part 170 and the depression 221b, and becomes detached from the socket 20 in conjunction with the open/close action of the cover 210.

### EMBODIMENT 3

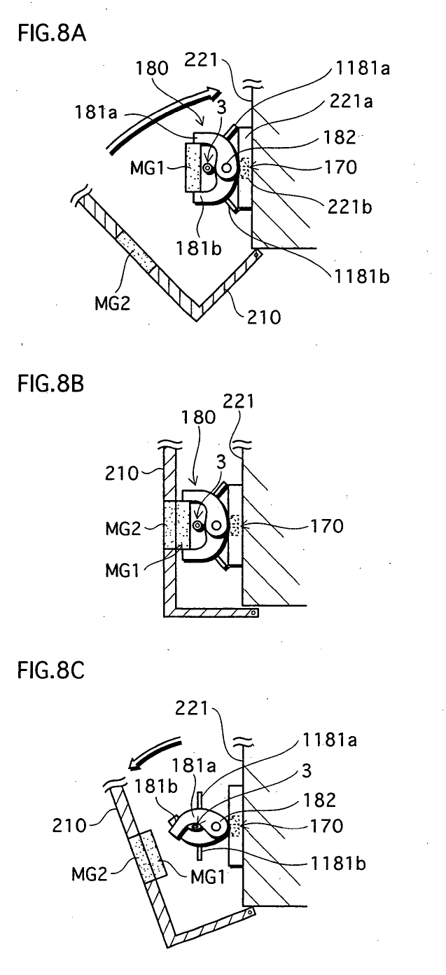

FIG. 8 shows a clamp set of Embodiment 3 of the present invention. Embodiment 3 differs from Embodiment 2 in using a scissor-type clamp (also called a hand clamp) 180 in place of the clamp set.

Specifically speaking, a pair of arms 181a and 181b biased by a publicly known torsion spring (not shown) in the direction that keeps the scissors closed are provided at the socket fitting position 221, and the arms 181 are fixed at the socket fitting position 221 by a linking part 182. On the paired arms 181a and 181b, operation parts 1811a and 1811b are provided so that the user is able to open the arms 181 manually. On the other hand, a magnet MG2 is set on the cover 210 at a position facing the scissor-type clamp 180. The clamp 180 is set using the same engaging part 170 and the depression 221b as those of Embodiment 2.

According to the structure above, the user first sandwiches a magnet MG 1 between the paired arms while the transfusion tube 3 is being inserted between the arms 181 of the scissor-type clamp 180 (FIG. 8A). In this state, the cover 210 is closed, and a steady state is achieved when magnets MG1 and MG2 stick together while the scissor-type clamp 180 remains in the open state (FIG. 8B).

In this state, if the cover 210 becomes unexpectedly opened, the state in which the magnet MG1 is attached to the magnet MG2 of the cover 210 is maintained, then the arms 181 of the scissor-type clamp 180 close as the transfusion tube 3 is kept inserted therein. Herewith, the transfusion tube 3 is firmly blocked off by the paired arms 181a and 181b (FIG. 8C).

According to the above operations, effects almost similar to those of Embodiment 2 can be realized. In the state shown in FIG. 8C, by using the operation parts 1811a and 1811b to nip the magnet MG1 with the paired arms 181a and 181b, the user can set the state again back to one shown in FIG. 8A.

Although Embodiment 3 uses the MG1 and MG2, the present invention is not limited to the case, similarly to Embodiment 2. Any structure can be used as long as an object sandwiched between the arms of the clamp engages with the cover so that the scissor-type clamp closes when the cover is opened. For example, a nonmagnetic object is placed between the arms of the clamp, and is then engaged with the cover using a hook and loop fastener, two-sided tapes, split-pin type projections or the like.

In addition, the scissor-type clamp is not limited to the structure using the above torsion spring, and a publicly known slide clamp may be used instead.

Although the cover 210 above is opened and closed with a hinge, it may have a structure that the cover 210 slides open and closed with respect to the case. Also in this case, the engaging parts of the socket and the clamp can be set on the cover and the clamp fitting position facing the cover.

### INDUSTRIAL APPLICABILITY

The present invention is applicable to medical transfusion pumps, and can also be used as a safety device for transfusion sets, such as an intravenous drip set.

1. A transfusion safety device comprising a clamp attached to a transfusion tube and a socket in which the clamp is stored in a freely detachable manner, wherein
the clamp has a structure in which fluid in the transfusion tube is released when the clamp is stored in the socket, and distribution of the fluid is blocked off when the clamp becomes detached from the socket, and
the socket has a structure in which the clamp becomes detached therefrom when an external force is applied to the transfusion tube.

2. The transfusion safety device of claim 1, wherein
the clamp includes:
an insertion path into which the transfusion tube is inserted; and
a movable mechanism that blocks off the distribution of the fluid by changing diameter of the insertion path.

3. The transfusion safety device of claims 2, wherein
the clamp includes:
a case having a 1st through-hole and a hollow part that communicates with the 1st through-hole;
a movable body which is slidably stored in the hollow part and has a 2nd through-hole; and
a bias unit that slidably biases the movable body in the hollow part, wherein
the movable mechanism causes the movable body to slide in the hollow part so that the 1st and 2nd through-holes overlap each other in an aperture direction thereof to form the insertion path, and changes the diameter of the insertion path by adjusting a degree of overlap between the 1st and 2nd through-holes with the use of the bias unit.

4. The transfusion safety device of claim 3, wherein
the clamp has a structure in which
the 1st through-hole and another 1st through-hole, which is paired with the 1st through-hole, are positioned on a lateral face of the case in a tubular shape having a base,
the movable body having the 2nd through-hole is stored in the hollow part of the case together with the bias unit, and
the movable body slides in a longitudinal direction of the case, which results in changing (i) length of the clamp in a longitudinal direction thereof and (ii) the diameter of the insertion path,
the socket includes paired locking walls whose distance therebetween is smaller than the length of the clamp detached from the socket, and
the clamp further has a structure in which the movable body is pushed toward the case against the bias unit, and stored between the paired locking walls of the socket with the 1st and 2nd through-holes being open.

5. The transfusion safety device of claim 3, wherein
the bias unit is a spring.

6. The transfusion safety device of claim 1, wherein
the clamp has one or more slits to guide the transfusion tube into an insertion path from a lateral side of the case.

7. A transfusion pump set, wherein
the transfusion safety device of claim 1 is attached to a transfusion pump.

8. The transfusion pump set of claim 7, wherein
the socket is attached to the transfusion pump with a higher strength than a pull force required to detach the clamp.

9. A transfusion safety device comprising a clamp attached to a transfusion tube and a socket in which the clamp is stored in a freely detachable manner, wherein
the clamp has a structure in which fluid in the transfusion tube is released when the clamp is stored in the socket, and distribution of the fluid is blocked off when the clamp becomes detached from the socket,
the socket is positioned on a cover which is freely open and closed and covers a predetermined area in a case of a transfusion pump, and
the clamp is provided in a manner that becomes detached from the socket in conjunction with opening of the cover.

10. The transfusion safety device of claim 9, wherein
the transfusion pump includes a 1st engaging body at the predetermined area,
the cover is fixed onto the case with a hinge,
the clamp includes a 2nd engaging body that is able to engage with the 1st engaging body when the cover is closed, and
engagement strength of the 1st and 2nd engaging bodies is set to be higher than strength required when the clamp becomes detached from the socket.

11. A transfusion safety device having a clamp attached to a transfusion tube, wherein
the clamp is a hand clamp having paired arms biased in a closing direction, and has a 1st engaging body held between tips of the paired arms in a manner that maintains the paired arms in an open direction,
the clamp is positioned, in a case of a transfusion pump, at a predetermined area enclosed by a cover that can be freely opened and closed,
a 2nd engaging body that can be engaged with the 1st engaging body is positioned on the cover at a location facing the clamp,
when the cover is closed, the 1st and 2nd engaging bodies engage with each other, and fluid in the transfusion tube inserted through the paired arms is released, and
the 1st engaging body becomes detached from the paired arms in conjunction with opening of the cover, which causes the paired arms to be in a closed state so that distribution of the fluid is blocked off.

12. The transfusion safety device of claim 10, wherein
the 1st and 2nd engaging bodies are made of a magnetic substance.

13. A transfusion pump set structured by the transfusion safety device of claim 9 being attached to a transfusion pump.
